(19) United States
(12) Patent Application Publication
(10) Pub. No.: US 2021/0208027 A1
(43) Pub. Date: Jul. 8, 2021

(54) ANALYSIS SYSTEM AND ANALYSIS METHOD

(71) Applicant: HORIBA, LTD., Kyoto (JP)

(72) Inventors: Takashi MATSUYAMA, Kyoto (JP); Ikue TAKAGI, Kyoto (JP)

(73) Assignee: HORIBA, LTD., Kyoto (JP)

(21) Appl. No.: 17/057,448

(22) PCT Filed: May 21, 2019
(86) PCT No.: PCT/JP2019/020068

§ 371 Date: Nov. 20, 2020

(30) Foreign Application Priority Data
May 22, 2018 (JP) 2018-098028

(51) Int. Cl. G01M 15/10; G01N 1/22; H01M 8/04

(52) U.S. Cl. CPC G01M 15/102; H01M 2250/20; H01M 8/04; G01N 1/22

(57) ABSTRACT

To enable exhaust gas analysis by taking into account the influence of particulates contained in air in a test chamber, an analysis system that analyzes exhaust gas discharged from a test piece that is provided in the test chamber and that includes an engine includes a sampling unit that samples the particulates contained in the air in the test chamber, a first analyzing unit that analyzes the particulates sampled by the sampling unit, and an analysis result output unit that outputs an analysis result of the first analyzing unit.

### TECHNICAL FIELD

The present invention relates to an analysis system for analyzing exhaust gas discharged from an engine, for example, and an analysis method therefor.

### Background Art

In the related art, as illustrated in PTL 1, for example, an analysis system dilutes exhaust gas from an engine with a dilution gas and samples the diluted exhaust gas. The analysis system analyzes components contained in the sampled diluted exhaust gas to measure the concentrations or masses of the components or to measure the weight, number, or the like of particulates contained in the diluted exhaust gas.

Air in a test chamber in which an engine is provided includes, for example, various particulates such as dust resulting from wear of a tire, a brake, or the like, dust generated from a rotational body, dust generated by an operator, and particulates generated from building materials of the test chamber.

To prevent such particulates from being mixed in the diluted exhaust gas to be analyzed or measured, the above-described analysis system uses a dilution air refiner that takes in the air in the test chamber through a filter or the like and refines dilution air, and uses the refined dilution air as the dilution gas.

However, the air in the test chamber is, for example, taken in to be supplied to the engine during driving of a vehicle, and thus, the exhaust gas discharged from the engine is supposed to contain the above-described particulates, and the particulates may possibly influence the analysis result.

Note that the particulates contained in the air in the test chamber are also supplied to the engine in, not only a case of using the above-described analysis system, but also in a case of analyzing the exhaust gas from the engine alone or of directly sampling the exhaust gas without dilution. Furthermore, the same also applies to a case of analyzing, for example, water discharged from a fuel cell vehicle that generates electricity by causing reaction between the air in the test chamber and hydrogen.

### CITATION LIST

### Patent Literature

PTL 1: Japanese Unexamined Patent Application Publication No. 6-3232

### SUMMARY OF INVENTION

### Technical Problem

The present invention has been made in order to solve the above-described problem, and a main object thereof is to enable exhaust gas analysis by taking into account the influence of the particulates contained in the air in the test chamber.

### Solution to Problem

That is, an analysis system according to the present invention is an analysis system that analyzes air in a test chamber in which a test piece, which is a vehicle or a component thereof, is provided and that includes: a sampling unit that samples particulates contained in the air in the test chamber; a first analyzing unit that analyzes the particulates sampled by the sampling unit; and an analysis result output unit that outputs an analysis result of the first analyzing unit.

Such an analysis system analyzes the particulates contained in the air in the test chamber. Thus, the mass or number of the particulates contained in the air can be known, or, for example, if titanium (Ti) is detected, it may be assumed that dust resulting from wear of a brake is contained in the air in the test chamber; and if a component (e.g., aluminum (Al)) used for building materials or the like of the test chamber is detected, it may be assumed that particles generated from the building materials or the like are contained in the air in the test chamber. This enables exhaust gas analysis by, for example, taking into account whether a particulate (hereinafter also referred to as contaminating substance) that may influence the exhaust gas analysis is contained in the air in the test chamber.

The analysis system preferably further includes: a second analyzing unit that analyzes an emission discharged from the test piece; and

a data storage unit that stores the analysis result of the first analyzing unit and an analysis result of the second analyzing unit in association with each other.

With such a configuration, after analysis is performed by the second analyzing unit, it is possible to analyze afterward whether a contaminating substance is contained in the air in the test chamber at the time of the analysis.

The analysis system preferably further includes: a dynamometer that places a load on the test piece; and an exhaust gas sampling device that samples exhaust gas discharged from the test piece, in which, while the exhaust gas sampling device is sampling the exhaust gas, the sampling unit preferably samples the particulates contained in the air in the test chamber.

With such an analysis system, since sampling of the exhaust gas and sampling of the air in the test chamber are synchronized with each other, if a contaminating substance is detected from the air in the test chamber, the contaminating substance may possibly be contained in the exhaust gas from an engine, which helps determination of the validity or the like of the exhaust gas analysis result.

The first analyzing unit preferably measures a mass or number of the particulates sampled by the sampling unit, and the second analyzing unit preferably analyzes components contained in the emission or measures a mass or number of the particulates contained in the emission.

With such a configuration, the second analyzing unit can perform various analyses (exhaust gas component analysis and particulates measurement) as necessary by taking into account how much particulates are contained in the air in the test chamber.

The first analyzing unit preferably performs elemental analysis of the particulates sampled by the sampling unit, and the second analyzing unit preferably analyzes components contained in the emission or measures a mass or number of the particulates contained in the emission.

With such a configuration, since elemental analysis of the particulates contained in the air in the test chamber is performed, the second analyzing unit can perform various analyses (exhaust gas component analysis and particulates measurement) as necessary by taking into account whether the element influences the analysis result of the second analyzing unit.

The analysis system preferably further includes: a source data storage unit that stores source estimation data in which a source of the particulates in the test chamber and an element contained in the source are associated with each other; and

a source estimating unit that estimates, on the basis of the analysis result of the first analyzing unit and the source estimation data, a source of the particulates contained in the air in the test chamber.

With such a configuration, the particulates contained in the air in the test chamber can be efficiently reduced by maintenance, cleaning, or the like of the estimated source.

The analysis system preferably further includes a validity determining unit that determines, on the basis of the analysis result of the first analyzing unit, validity of the analysis result of the second analyzing unit.

With such a configuration, it is possible to determine the validity of the exhaust gas analysis result by quantitatively taking into account the particulates contained in the air in the test chamber.

The sampling unit preferably has a sampling port provided near an intake port of an engine of the test piece.

With such a configuration, it is possible to directly perform elemental analysis of the air to be taken in the engine and more accurately determine whether a contaminating substance is supplied to the engine.

Furthermore, an analysis method according to the present invention is an analysis method that analyzes exhaust gas discharged from air in a test chamber in which a test piece, which is a vehicle or a component thereof, is provided and that includes: a sampling step for sampling particulates contained in the air in the test chamber; a first analyzing step for analyzing the particulates sampled by the sampling unit; and an analysis result outputting step for outputting an analysis result of the first analyzing step.

Such an analysis method can produce substantially the same effects as those of the above-described analysis system.

### Advantageous Effects of Invention

The present invention having the above configuration enables exhaust gas analysis by taking into account the influence of the particulates contained in the air in the test chamber.

### BRIEF DESCRIPTION OF DRAWINGS

FIG. 1 is an overall schematic diagram of an analysis system according to this embodiment.

FIG. 2 schematically illustrates a configuration of an environment analysis device according to the same embodiment.

FIG. 3 is a functional block diagram illustrating functions of an information processing device according to the same embodiment.

FIG. 4 is a flowchart illustrating an operation of the analysis system according to the same embodiment.

FIG. 5 is a functional block diagram illustrating functions of an information processing device according to another embodiment.

FIG. 6 is an overall schematic diagram of an analysis system according to another embodiment.

### DESCRIPTION OF EMBODIMENTS

Now, an embodiment of an analysis system according to the present invention will be described with reference to the drawings.

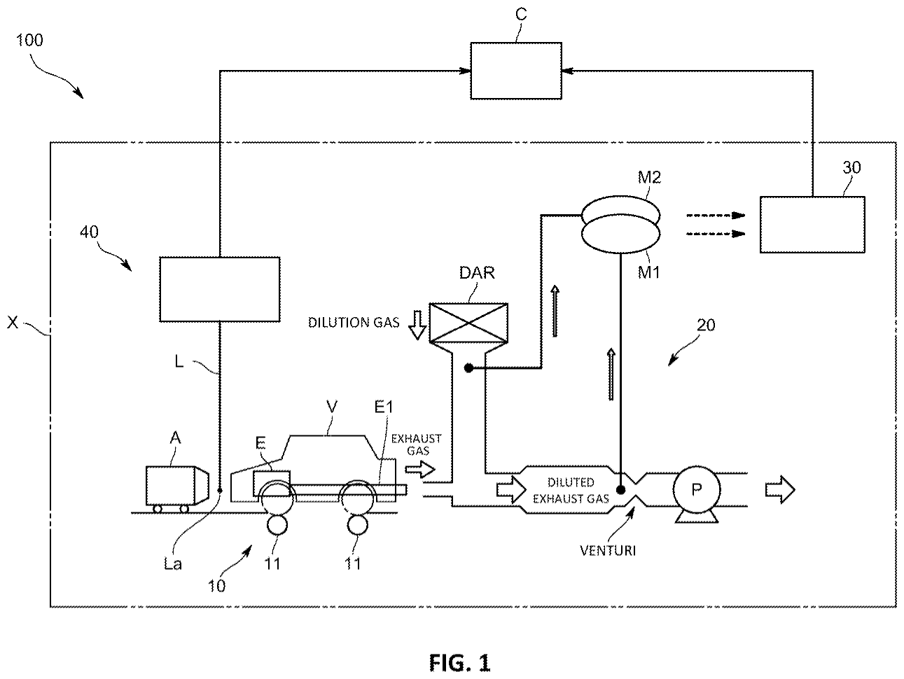

An analysis system 100 according to this embodiment is an exhaust gas analysis system to be used to evaluate the performance or the like of a test piece including an engine E. As illustrated in FIG. 1, in a test chamber X called cell, the analysis system 100 analyzes exhaust gas discharged from the engine E. Note that the exhaust gas analysis means to analyze components contained in the exhaust gas to measure the concentrations or masses of the components or to measure the PM (mass), PN (number), or the like contained in the exhaust gas. In addition, the engine is an internal combustion engine or an external combustion engine used for a vehicle, a ship, an aircraft, or the like. Note that the test piece including the engine E is a concept including the engine E alone. In addition, as long as including a rotational body, the test piece does not necessarily include the engine E and may be, for example, a fuel cell vehicle (hereinafter, FCV), an electric vehicle (hereinafter, EV), or a component thereof. Furthermore, the test piece may be a hybrid vehicle (hereinafter, HV) including both an engine and a motor. Furthermore, the analysis target of the analysis system 100 is not limited to exhaust gas but may be any emission that is discharged from the test piece, such as water discharged from a tailpipe.

The following description will illustrate an embodiment in which a test vehicle V equipped with the engine E is run on a chassis dynamometer 10 for exhaust gas analysis. However, the analysis system 100 may perform the exhaust gas analysis by connecting an engine dynamometer to the engine E alone or by connecting a dynamometer to a power train to which the engine E is connected.

The analysis system 100 according to this embodiment includes the chassis dynamometer 10 that places a load on the test vehicle V, an exhaust gas sampling device 20 that samples the exhaust gas discharged from the engine E, and an exhaust gas analyzing device 30 (second analyzing unit in the claims) that analyzes the exhaust gas sampled by the exhaust gas sampling device 20.

The chassis dynamometer 10 includes a rotating drum 11 on which driving wheels of the test vehicle V are to be placed, for example. Although the rotating drum 11 is provided for both front wheels and rear wheels of the test vehicle V in this embodiment, the rotating drum 11 may also be provided for either one of the front wheels and the rear wheels.

The exhaust gas sampling device 20 samples part or all of the exhaust gas flowing through an exhaust pipe E1 connected to the engine E. The exhaust gas sampling device 20 according to this embodiment includes a constant volume sampling (CVS) mechanism that is configured to sample all of the exhaust gas and also to generate a diluted exhaust gas by diluting the all-sampled exhaust gas with a dilution gas so that the flow rate of the diluted exhaust gas becomes constant. With this CVS mechanism, in a state where the total flow rate of the exhaust gas and the dilution gas, that is, the flow rate of the diluted exhaust gas, is constant, the diluted exhaust gas is accommodated in a first gas accommodating bag M1, and also the dilution gas is accommodated in a second gas accommodating bag M2. Note that the dilution gas is air for dilution obtained by an air refiner DAR taking in and refining air in the test chamber X through a filter, which is not illustrated.

The exhaust gas analyzing device 30 analyzes components contained in the exhaust gas to measure the concentrations and/or masses of the components. Specifically, the exhaust gas analyzing device 30 analyzes the gases accommodated in the accommodating bags M1 and M2, measures the concentration of measurement target components such as HC, NO_{X}, CO, and C₂ , contained in the gases, and, on the basis of the measurement result, calculates the concentration of the measurement target components contained in the exhaust gas discharged from the engine E.

The analysis system 100 according to this embodiment further includes an environment analysis device 40 and an information processing device C. The environment analysis device 40 analyzes the environment in which the exhaust gas analysis is performed, that is, the air in the test chamber X. The information processing device C acquires the above-described exhaust gas analysis result and the analysis result of the environment analysis device 40.

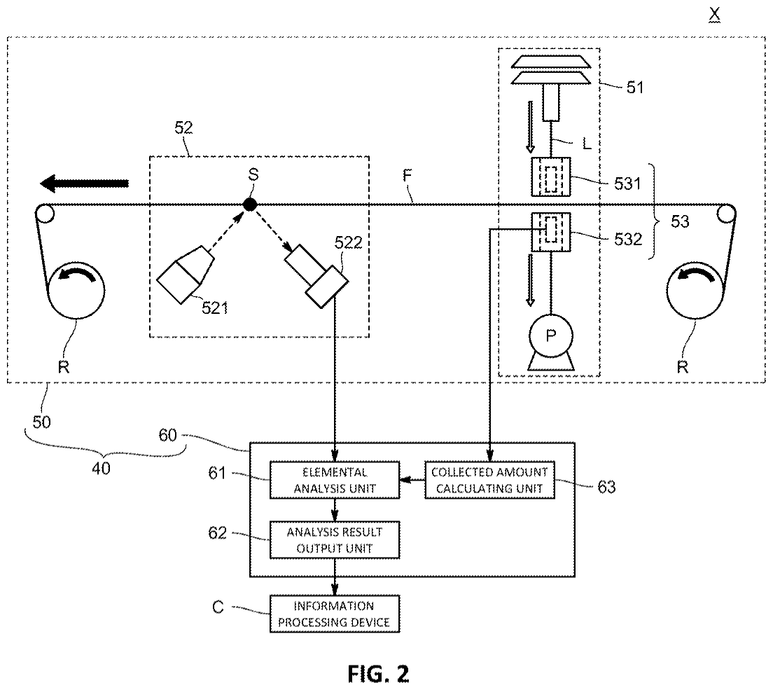

As illustrated in FIG. 2, the environment analysis device 40 irradiates a sample S with a primary X-ray and detects a resulting secondary X-ray to analyze components contained in the sample S. Specifically, the environment analysis device 40 is a fluorescent X-ray analyzer.

The environment analysis device 40 herein is, for example, wavelength dispersive fluorescent X-ray analyzer that is provided in the test chamber X to analyze, as the sample S, particulates contained in the air in the test chamber X, and includes a device main body 50 and a data processing unit 60 that transmits or receives a signal to or from the device main body 50.

The device main body 50 includes a sampling unit 51 and an analyzing unit 52. The sampling unit 51 samples particulates contained in the air in the test chamber X. The analyzing unit 52 irradiates the sampled particulates with a primary X-ray and detects a fluorescent X-ray generated from the particulates.

The sampling unit 51 includes a filter F for collecting the particulates, a sampling line L for guiding the air sucked from the test chamber X to the filter F, and a suction pump P provided in the sampling line L. More specifically, the sampling unit 51 according to this embodiment is of a winding type that winds the filter F by using a pair of reels R, and is configured to, after collecting the particulates in the filter F over a predetermined sampling time, send the filter F to the analyzing unit 52. Note that the sampling unit 51 may also be of a batch type for which, after collecting the particulates, a user removes the filter F from the sampling line L and sets it for the analyzing unit 52.

The sampling unit 51 is provided in the test chamber X so as to sample part of the air to be taken in the test vehicle V. Specifically, as illustrated in FIG. 1, a sampling port La of the sampling line L is preferably provided in front of the test vehicle V and near an intake port of the test vehicle V. In this embodiment, a blower fan A is provided in front of the test vehicle V, and the sampling port La is provided between the test vehicle V and the blower fan A.

The sampling unit 51 further includes a collected amount measuring unit 53 for measuring a collected amount (mass) of the particulates collected in the filter F. The collected amount measuring unit 53 includes a β-ray source 531 and a β-ray detector 532. The β-ray source 531 irradiates the collected particulates with a β-ray. The β-ray detector 532 detects the β-ray transmitted through the particulates. Thus, on the basis of the β-ray intensity detected by the P-ray detector 532, the collected amount of the particulates can be obtained. Note that the collected amount of the particulates may also be obtained by subtracting the mass of the filter F before collection from the mass or the filter F after collection.

The analyzing unit 52 includes an X-ray source 521 and an X-ray detector 522. The X-ray source 521 irradiates the particulates collected in the filter F with an X-ray. The X-ray detector 522 detects a fluorescent X-ray generated from the particulates. For example, the X-ray source 521 may be one that generates an X-ray by irradiating a metal such as palladium with electrons. For example, the X-ray detector 522 may be a silicon semiconductor detector or a silicon drift detector.

The data processing unit 60 physically includes a CPU, an internal memory, an input/output interface, an AD converter, and the like and has a function of an elemental analysis unit 61, an analysis result output unit 62, and the like. On the basis of an inspection program stored in the internal memory, in collaboration with the CPU and other structural elements, the elemental analysis unit 61 analyzes elements contained in the particulates. The analysis result output unit 62 outputs the elemental analysis result of the elemental analysis unit 61 (first analyzing unit in the claims).

The elemental analysis unit 61 acquires the X-ray intensity signal output from the above-described X-ray detector 522 and performs at least qualitative analysis of elements contained in the particulates. Specifically, the elemental analysis unit 61 identifies an element corresponding to a peak in the detected fluorescent X-ray spectrum.

The data processing unit 60 according to this embodiment further includes a function of a collected amount calculating unit 63 that acquires the β-ray intensity signal output from the above-described β-ray detector 532 and calculates the collected amount. Thus, by using the collected amount calculated by the collected amount calculating unit 63, the elemental analysis unit 61 can perform quantitative analysis of the concentrations (e.g., mass concentration or element concentration) or masses of elements contained in the particulates.

The analysis result output unit 62 outputs the elemental analysis result of the elemental analysis unit 61 to the information processing device C. The output elemental analysis result includes at least the qualitative analysis result, that is, information of elements determined to be included in the particulates. Furthermore, as the elemental analysis result, the analysis result output unit 62 herein further outputs the quantitative analysis result, that is, information indicating the concentration (e.g., mass concentration or element concentration) or mass of the determined elements.

Note that as the output method of the analysis result output unit 62, the elemental analysis result may be output to the information processing device C with or without wires, or, for example, the elemental analysis result may be output to an external memory, such as a USB memory, and the information processing device C may be caused to acquire the elemental analysis result stored in the external memory at appropriate timing.

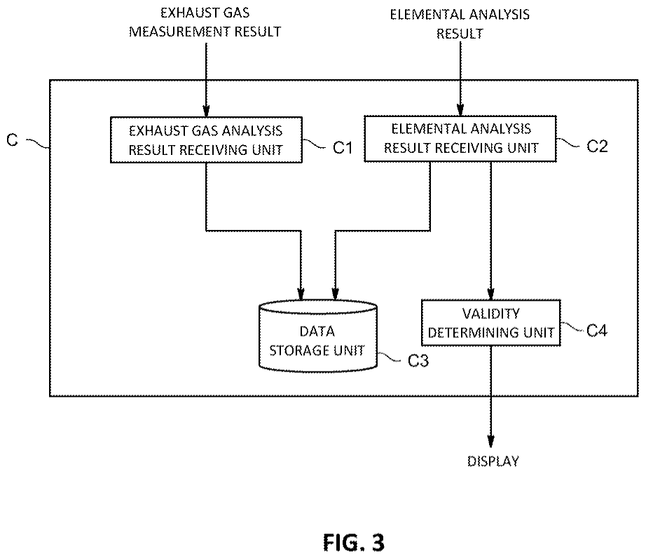

The information processing device C is a dedicated or general-purpose computer including a CPU, an internal memory, an input/output interface, an AD converter, and the like, and is, for example, provided in a chamber different from the test chamber X. On the basis of a program stored in the internal memory, as illustrated in FIG. 3, in collaboration with the CPU and other structural elements, the information processing device C is configured to implement, functions of an exhaust gas analysis result receiving unit C1, an elemental analysis result receiving unit C2, a data storage unit C3, a validity determining unit C4, and the like.

Now, each of the units will be described below.

The exhaust gas analysis result receiving unit C1 receives the exhaust gas analysis result obtained by the above-described exhaust gas analyzing device 30. The exhaust gas analysis result receiving unit C1 receives components contained in the exhaust gas, the concentrations and/or masses of the components, PM or PN contained in the exhaust gas, or the like.

The elemental analysis result receiving unit C2 receives the elemental analysis result output from the analysis result output unit 62. The elemental analysis result receiving unit C2 receives at least the qualitative analysis result obtained through qualitative analysis of the particulates contained in the air in the test chamber X. Herein, the elemental analysis result receiving unit C2 also receives the quantitative analysis result obtained through quantitative analysis of the particulates.

The data storage unit C3 stores the exhaust gas analysis result received by the exhaust gas analysis result receiving unit C1 and the elemental analysis result received by the elemental analysis result receiving unit C2 in association with each other. Specifically, for example, the data storage unit C3 stores the exhaust gas analysis result and the elemental analysis result in synchronization with each other such that a sampling time slot of the exhaust gas by the exhaust gas sampling device 20 and a sampling time slot of the air in the test chamber X by the sampling unit 51 at least partly overlap with each other. In other words, the data storage unit C3 stores the exhaust gas analysis result and the elemental analysis result of the air in the test chamber X in association with each other, the air being sampled from the sampling start time of the analyzed exhaust gas until the sampling end time of the exhaust gas.

Note that the data storage unit C3 may also store, for example, the exhaust gas analysis result and the elemental analysis result in synchronization with each other such that the analysis time slot of the sampled exhaust gas and the analysis time slot of the sampled air in the test chamber X at least partly overlap with each other.

On the basis of the elemental analysis result output from the analysis result output unit 62, the validity determining unit C4 determines the validity of the exhaust gas analysis result. Specifically, from element information included in the elemental analysis result, that is, from among the elements detected through qualitative analysis, the validity determining unit C4 compares the concentration or mass of at least one predetermined element (hereinafter referred to as contaminating element) determined by a user in advance with a threshold (including zero) that is defined for the corresponding one of predetermined elements. If the concentration or mass of the contaminating element is less than or equal to the threshold, the validity determining unit C4 determines that the exhaust gas analysis result is valid; if the concentration or mass of the contaminating element exceeds the threshold, the validity determining unit C4 determines that the exhaust gas analysis result is invalid. The validity determining unit C4 outputs the determination result to, for example, a display or the like. Note that the validity determining unit C4 may also be configured to, for example, output the elemental analysis result (the concentration or mass of the contaminating element) to a display or the like so as to be compared with the threshold without outputting the determination result.

The contaminating element herein is an element constituting the particulates that may be contained in the air in the test chamber X and that influence the exhaust gas analysis if supplied to the engine E, for example. Specifically, for example, the contaminating element may be an element contained in dust resulting from wear of a tire, brake, or the like, dust generated from a rotational body, dust generated by an operator, particles generated from building materials of the test chamber X, or the like. More specifically, the contaminating element may be titanium (Ti) contained in a brake, aluminum (Al) contained in building materials of the test chamber X or the like, iron (Fe) contained in a rotational body of, for example, the rotating drum 11 constituting the chassis dynamometer 10, or the like. However, the contaminating element may be an element that does not influence the exhaust gas analysis as necessary, and a user may select or change the contaminating element as appropriate.

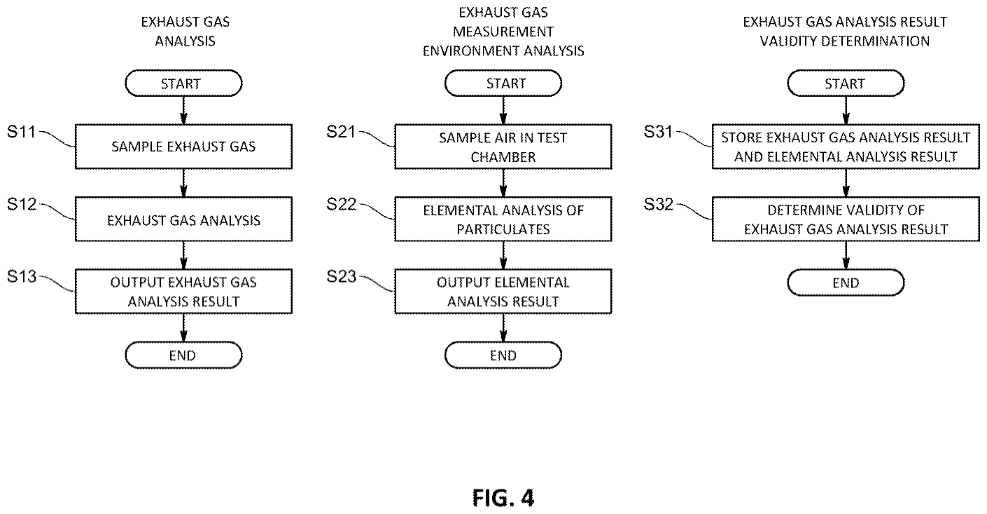

Next, an analysis method using the environment analysis device 40 according to this embodiment will be described with reference to FIG. 4.

First, when the exhaust gas analysis starts, the exhaust gas sampling device 20 samples the exhaust gas discharged from the engine E (S11). In this embodiment, the exhaust gas is diluted with a dilution gas, the diluted exhaust gas is accommodated in the first gas accommodating bag M1, and the dilution gas is accommodated in the second gas accommodating bag M2.

On the other hand, when the diluted exhaust gas and the dilution gas are accumulated in the gas accommodating bags M1 and M2, respectively, the exhaust gas analyzing device 30 analyzes components contained in the gasses and measures the concentrations and/or masses of the components, the PM or PN thereof, or the like (S12). The exhaust gas analysis result is output from the exhaust gas analyzing device 30 to the information processing device C (S13).

Before or concurrently with the timing at which sampling the above-described exhaust gas starts, the sampling unit 51 of the environment analysis device 40 starts to sample the air in the test chamber X (S21). Thus, while the above-described exhaust gas is being sampled, the air in the test chamber X is sampled, and particulates contained in the sampled air are collected in the filter F.

Subsequently, when a predetermined sampling time elapses, the filter F is sent to the analyzing unit 52. The collected particulates are irradiated with a primary X-ray, a resulting fluorescent X-ray is detected by the X-ray detector 522, and on the basis of the detected X-ray intensity signal, the data processing unit 60 performs elemental analysis of the particulates (S22). The elemental analysis result is output from the data output unit to the information processing device C (S23).

The information processing device C receives the above-described elemental analysis result and the exhaust gas analysis result, and stores these results in association with each other in the data storage unit C3 (S31). Herein, as described above, the data storage unit C3 stores the exhaust gas analysis result and the elemental analysis result in synchronization with each other such that the sampling time of the exhaust gas and the sampling time of the air in the test chamber X at least partly overlap with each other.

Upon the elemental analysis result being output to the information processing device C, on the basis of the elemental analysis result, the validity determining unit C4 determines the validity of the exhaust gas analysis result (S32). Note that the specific method for determination by the validity determining unit C4 is described above.

Regarding the timing of determination by the validity determining unit C4, the validity may be determined for an exhaust gas analysis result after the exhaust gas analysis result is obtained, or the validity may be determined, before an exhaust gas analysis result is obtained, for the exhaust gas analysis result to be obtained if the exhaust gas analysis is continued. In the latter case, for example. the validity determining unit C4 may be configured to determine the validity while the exhaust gas is being sampled, on the basis of the elemental analysis result output from the environment analysis device 40, for the exhaust gas analysis result to be obtained if the exhaust gas analysis is continued.

The analysis system 100 having such a configuration can analyze elements of particulates contained in the air in the test chamber X. Thus, for example, if Ti is detected, it may be assumed that dust resulting from wear of a brake is contained in the air in the test chamber X; and if Al used for building materials or the like of the test chamber X is detected, it may be assumed that, particles generated from the building materials or the like are contained in the air in the test chamber X. This enables prosecution of the exhaust gas analysis or determination of the validity of the obtained exhaust gas analysis result by, for example, taking into account whether a contaminating element that may influence the exhaust gas analysis is contained in the air in the test chamber X.

In addition, sampling of the exhaust gas and sampling of the air in the test chamber X are synchronized with each other. Thus, if a contaminating element is detected from the air in the test chamber X, the contaminating element may possibly be contained in the exhaust gas from the engine E, which helps determination of the validity or the like of the exhaust gas analysis.

Furthermore, the exhaust gas analysis result and the elemental analysis result are stored in association with each other. Thus, after the exhaust gas analysis, it is possible to analyze afterward whether a contaminating element is contained in the air in the test chamber X at the time of the exhaust gas analysis.

Furthermore, the validity determining unit C4 determines the validity of the exhaust gas analysis result on the basis of the concentration or mass of a predetermined element that influences the exhaust gas analysis. This enables determination of the validity of the exhaust gas analysis result by quantitatively taking into account the contaminating element contained in the air in the test chamber X.

Besides, the sampling port La of the sampling line L for sampling the air in the test chamber X is provided near the intake port of the test vehicle V. This enables direct elemental analysis of the air to be taken in the engine E and more accurate determination as to whether a contaminating substance is supplied to the engine E.

Furthermore, if the validity determining unit C4 is configured to determine the validity, during the exhaust gas is being sampled, for an exhaust gas analysis result to be obtained if the exhaust gas analysis is continued, in a case where, for example, a large number of contaminating elements are contained in the air in the test chamber X and the validity of the analysis result to be obtained if the exhaust gas analysis is continued is low, for example, the sampling of the exhaust gas may be stopped so as to avoid useless exhaust gas analysis.

Note that the present invention is not limited to the above embodiment.

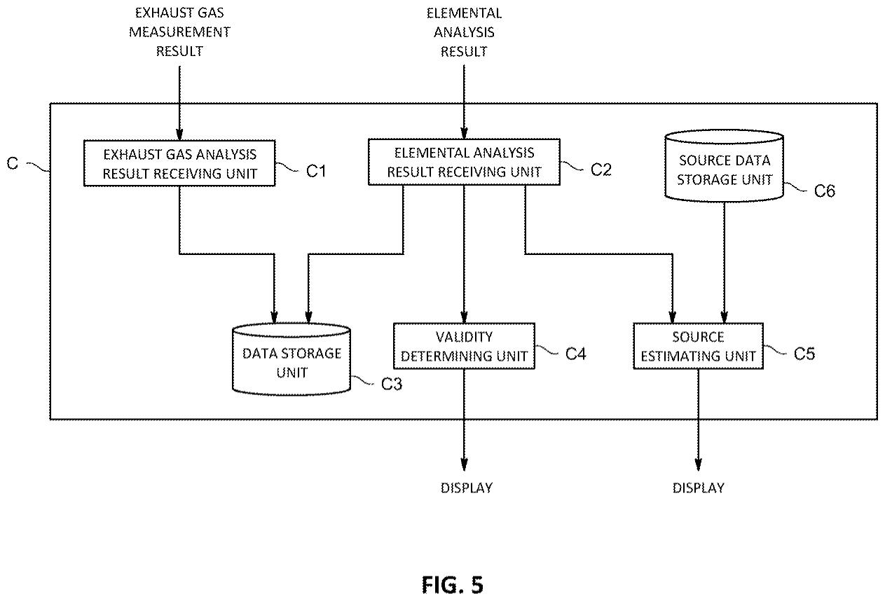

For example, as illustrated in FIG. 5, the information processing device C may further include a function of a source estimating unit C5 that acquires the elemental analysis result, and, if the elemental analysis result Includes a contaminating element that influences the exhaust gas analysis, estimates the source of the contaminating element in the test chamber X.

Specifically, the information processing device C in this case further includes a function of a source data storage unit C6 that stores source estimation data in which a contaminating element that influences the exhaust gas analysis and a source that may generate the contaminating element in the test chamber X are associated with each other. The source estimating unit C5 is configured to estimate the source on the basis of the source estimation data and the elemental analysis result, and output the source to, for example, a display or the like.

With such a configuration, the contaminating elements contained in the test chamber X can be reduced by cleaning or maintenance of the source estimated by the source estimating unit C5.

The data storage unit C3 according to the above embodiment stores the exhaust gas analysis result and the elemental analysis result of the air in the test chamber X, sampled in the sampling time slot of the analyzed exhaust gas, in association with each other. However, the sampling time slot of the exhaust gas and the sampling time slot of the air may not overlap with each other, and the data storage unit C3 may store analysis result of the exhaust gas and the elemental analysis result of the air, the exhaust gas and the air being sampled in sampling time slots different from each other.

In addition, in the above embodiment, the validity of the exhaust gas analysis result is determined on the basis of the elemental analysis result obtained by the environment analysis device 40. However, for example, by obtaining the elemental analysis result before start of the exhaust gas analysis, on the basis of the elemental analysis result, it may be determined whether an exhaust gas analysis environment needs to be cleaned, for example, before start of the exhaust gas analysis.

Furthermore, in the above embodiment, the environment analysis device 40 performs elemental analysis by irradiating the sample S with a primary X-ray and detecting a resulting fluorescent X-ray. However, the environment analysis device 40 may perform elemental analysis by detecting scattered X-rays or photoelectrons generated by irradiation with a primary x-ray. In addition, the fluorescent X-ray analyzer to be used as the environment analysis device 40 is not limited to a wavelength dispersive type but may be an energy dispersive type.

In addition, the first analyzing unit according to the above embodiment performs elemental analysis of particulates contained in the air in the test chamber X. However, the first analyzing unit may measure the mass or number of particulates contained in the air in the test chamber X.

The first analyzing unit in this case may be a collection filter for measuring the mass of particulates (PM) or a particle counter for measuring the number of particulates (PN) using a diffusion charge sensor (DCS), a condensation particle counter (CPC), a solid particle counting system (SPCS), or the like.

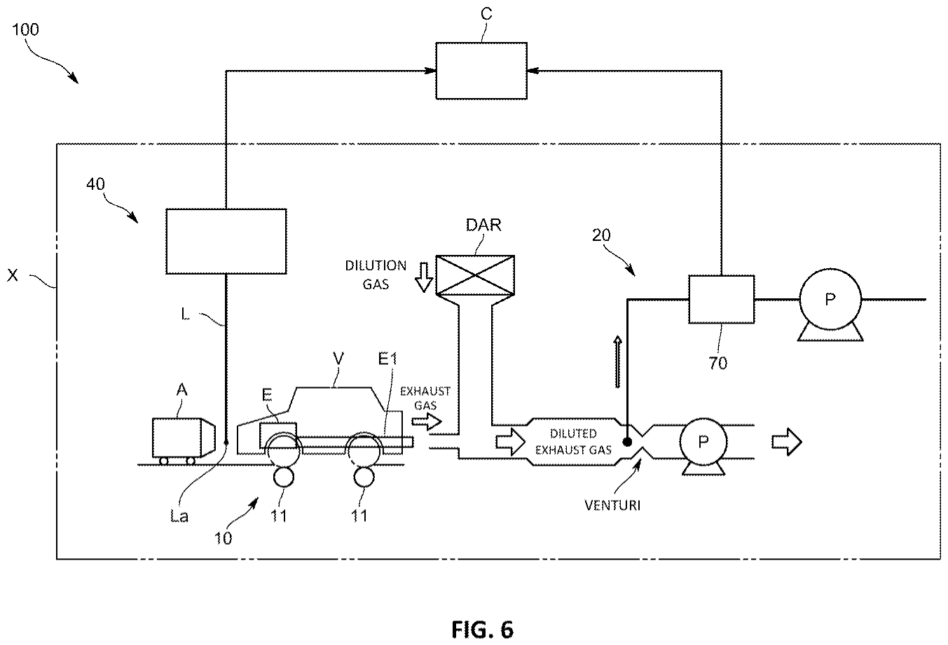

Furthermore, the second analyzing unit according to the above embodiment analyzes components contained in the exhaust gas sampled by the exhaust gas sampling device 2. However, as illustrated in FIG. 6, the second analyzing unit may be a particulates measuring device 70 that measures the mass or number of the particulates contained in the exhaust gas sampled by the exhaust gas sampling device 2.

In this case, the particulates measuring device 70 may be, a PM measuring device using a diffusion charge sensor (DCS) for measuring the mass of particulates (PM), a collection filter for collecting particulates, or a particle counter for measuring the number of particulates (PN) using a condensation particle counter (CPC), a solid particle counting system (SPCS), or the like.

Besides, in the above embodiment, the analysis result output unit 62 outputs the elemental analysis result to the information method processing device. However, the analysis result output unit 62 may also output and display the elemental analysis result on, for example, a display, or may output and print the elemental analysis result on paper. Also with such a configuration, by a user checking the elemental analysis result, exhaust gas analysis can be performed by taking into account the influence of particulates contained in the air in the test chamber X.

Furthermore, in the above embodiment, the analysis system 100 samples all of the exhaust gas discharged from the engine E and dilutes it for analysis. However, the analysis system 100 may also sample part of the exhaust gas discharged from the engine E. In addition, the analysis system 100 may also directly sample and analyze the exhaust gas discharged from the engine E without dilution.

In addition, the test piece of the analysis system 100 may be an FCV, an EV, an HV, a two-wheeled vehicle, or the like, or may be a component thereof.

Note that the FCV generates water when generating electricity by causing reaction between hydrogen and air (e.g., compressed air) in a test chamber. Thus, if the FCV is the test piece, in place of the exhaust gas analyzing device 30 according to the above embodiment, the second analyzing unit may be an analyzing device (e.g., elemental analysis device) that analyzes water discharged from a tailpipe of the FCV. With such a configuration, water discharged from the tailpipe can be analyzed by taking into account the influence of particulates contained in air in the test chamber. For example, the information processing device can identify particulates resulting from a fuel cell by subtracting the analysis result of the first analyzing unit (i.e., particulates contained in the air in the test chamber) from the water analysis result of the second analyzing unit. Furthermore, the electrical efficiency of the FCV may be measured, and the information processing device may store the analysis result (PM or PN) of particulates contained in the air in the test chamber and the electrical efficiency of the FCV in association with each other, or may store particulates resulting from the fuel cell and the electrical efficiency of the FCV in association with each other.

Thus, the correlation between the PM or PN in the test chamber and the electrical efficiency can be obtained.

Besides, it is needless to say that the present invention is not limited to the above embodiment, and any modification may be made without departing from the gist thereof.

### REFERENCE SIGNS LIST

100 analysis system
E engine
E1 exhaust pipe
X test chamber
V test vehicle
10 chassis dynamometer
11 rotating drum
20 exhaust gas sampling device 30 exhaust gas analyzing device
40 environment analysis device
S sample
50 device main body
51 sampling unit
F filter
L sampling line
P suction pump
R reel
La sampling port
52 analyzing unit
521 X-ray source
522 X-ray detector
53 collected amount measuring unit
531 p-ray source
532 p-ray detector
60 data processing unit
61 elemental analysis unit
62 analysis result output unit
63 collected amount calculating unit
C information processing device
C1 exhaust gas analysis result receiving unit
C2 elemental analysis result receiving unit
C3 data storage unit
C4 validity determining unit
C5 source estimating unit.
C6 source data storage unit

### INDUSTRIAL APPLICABILITY

The present invention enables exhaust gas analysis taking into account the influence of the particulates contained in the air in the test chamber.

1. An analysis system that analyzes air in a test chamber in which a test piece, which is a vehicle or a component thereof, is provided, the analysis system comprising:
a sampling unit that samples particulates contained in the air in the test chamber;
a first analyzing unit that analyzes the particulates sampled by the sampling unit; and
an analysis result output unit that outputs an analysis result of the first analyzing unit.

2. The analysis system according to claim 1, further comprising:
a second analyzing unit that analyzes an emission discharged from the test piece; and
a data storage unit that stores the analysis result of the first analyzing unit and an analysis result of the second analyzing unit in association with each other.

3. The analysis system according to claim 1, further comprising:
a dynamometer that places a load on the test piece; and
an exhaust gas sampling device that samples exhaust gas discharged from the test piece,
wherein, while the exhaust gas sampling device is sampling the exhaust gas, the sampling unit samples the particulates contained in the air in the test chamber.

4. The analysis system according to claim 2,
wherein the first analyzing unit measures a mass or number of the particulates sampled by the sampling unit, and
wherein the second analyzing unit analyzes components contained in the emission or measures a mass or number of the particulates contained in the emission.

5. The analysis system according to claim 2,
wherein the first analyzing unit performs elemental analysis of the particulates sampled by the sampling unit, and
wherein the second analyzing unit analyzes components contained in the emission or measures a mass or number of the particulates contained in the emission.

6. The analysis system according to claim 5, further comprising:
a source data storage unit that stores source estimation data in which a source of the particulates in the test chamber and an element contained in the source are associated with each other; and
a source estimating unit that estimates, on the basis of the analysis result of the first analyzing unit and the source estimation data, a source of the particulates contained in the air in the test chamber.

7. The analysis system according to claim 4, further comprising:
a validity determining unit that determines, on the basis of the analysis result of the first analyzing unit, validity of the analysis result of the second analyzing unit.

8. The analysis system according to claim 1,
wherein the sampling unit has a sampling port provided near an intake port of an engine of the test piece.

9. An analysis method that analyzes air in a test chamber in which a test piece, which is a vehicle or a component thereof, is provided, the analysis method comprising:
a sampling step for sampling particulates contained in the air in the test chamber;
a first analyzing step for analyzing the particulates sampled by the sampling unit; and
an analysis result outputting step for outputting an analysis result of the first analyzing step.
